(19) United States
(12) Patent Application Publication
(10) Pub. No.: US 2024/0260940 A1
(43) Pub. Date: Aug. 8, 2024

(54) MEDICAL SYSTEM AND DATA PROCESSING METHOD

(71) Applicant: FUJIFILM Healthcare Corporation, Kashiwa-shi (JP)

(72) Inventors: Takanori Hirai, Chiba (JP); Masami Noda, Chiba (JP)

(21) Appl. No.: 18/428,163

(22) Filed: Jan. 31, 2024

(30) Foreign Application Priority Data
Feb. 8, 2023 (JP) 2023-017823

(51) Int. Cl. A61B 8/00

(52) U.S. Cl. CPC A61B 8/463; A61B 8/4416; A61B 8/4477; A61B 8/565

(57) ABSTRACT

A system can be configured to reduce a burden on an examiner in a case in which a past image and a current image are displayed in an ultrasound examination. In an ultrasound examination of a first time, a file creation unit creates an information file including volume data specification information and positional information. In an ultrasound examination of a second time, volume data is specified based on the volume data specification information, and a position of an observation cross section set for the volume data is specified based on the positional information. An image processing unit generates a tomographic image representing the observation cross section as a past image. An ultrasound image formation unit forms a real-time ultrasound tomographic image representing the observation cross section as a current image.

### CROSS-REFERENCE TO RELATED APPLICATION

The present application claims priority from Japanese patent application 2023-017823 filed with the Japanese Patent Office on Feb. 8, 2023, the content of which is hereby incorporated by reference into this application.

### BACKGROUND OF THE INVENTION

### 1. Field of the Invention

The present disclosure relates to a medical system and a data processing method, and more particularly to a technique of simultaneously displaying a past image and a current image.

### 2. Description of the Related Art

A medical system is configured with, for example, an ultrasound diagnostic apparatus, a magnetic resonance imaging (MRI) apparatus, and a server. For example, data acquired by the MRI apparatus is uploaded to the server, and the data is downloaded from the server to the ultrasound diagnostic apparatus. A CT apparatus or the like may be provided in the medical system. Hereinafter, an image generated from volume data acquired by the MRI apparatus will be referred to as an MR image.

For example, in an examination of a mammary gland, an MM examination is first performed, and an affected part is specified through observation of the MR image based on the volume data obtained from the MRI examination. An ultrasound examination of the mammary gland is performed with or after the MRI examination. In a case in which chemotherapy is selected as a treatment method for the affected part, the ultrasound examination of the mammary gland is repeatedly performed over a long period.

In the ultrasound examination, in general, an image comparison function provided in the ultrasound diagnostic apparatus is used. This function is to simultaneously display a past tomographic image (past image) and a current tomographic image (current image) in parallel. The latter current image is a real-time ultrasound tomographic image. The former past image is, for example, an MR image generated based on MRI volume data acquired in the past. The MR image is typically an image showing a state of the affected part before or at the start of the treatment.

Prior to the display of the MR image on the ultrasound diagnostic apparatus, the volume data is transmitted from the server to the ultrasound diagnostic apparatus. Thereafter, an observation cross section is set for the volume data. The cross section data corresponding to the observation cross section is cut out from the volume data, and the MR image is generated based on the cross section data. A content of the MR image is changed with a change of the observation cross section. In a case in which a user is requested to designate the same observation cross section each time the ultrasound examination for a follow-up observation is performed, there may be a problem that a burden on the user is increased or the observation cross section is not set correctly.

JP2008-188163A, JP2010-88587A, and JP2011-11001A disclose a medical system including an ultrasound diagnostic apparatus. JP2008-188163A, JP2010-88587A, and JP2011-11001A do not disclose a technique of repeatedly using information for specifying an observation cross section over a plurality of ultrasound examinations.

### SUMMARY OF THE INVENTION

An object of the present disclosure is to reduce a burden on an examiner who is a user in a case in which a past image and a current image are displayed in an ultrasound examination. Alternatively, an object of the present disclosure is to display a correct past image in a case in which the past image and the current image are displayed in the ultrasound examination. Alternatively, an object of the present disclosure is to achieve information sharing among a plurality of ultrasound diagnostic apparatuses.

An aspect of the present disclosure relates to a medical system including: a first processing unit configured to, in a first ultrasound examination on a subject, extract first cross section data corresponding to a first observation cross section from volume data acquired from an inside of the subject, generate and display a first past image as a tomographic image based on the first cross section data, and display a first current image as a real-time ultrasound tomographic image representing the first observation cross section; a transmission unit configured to, in or after the first ultrasound examination, transmit an information file including positional information of the first observation cross section to a server; an acquisition unit configured to, in a second ultrasound examination after the first ultrasound examination, acquire the information file from the server; and a second processing unit configured to, in the second ultrasound examination, set a second observation cross section for the volume data based on the positional information in the information file, extract second cross section data corresponding to the second observation cross section from the volume data, generate and display a second past image based on the second cross section data, and display a second current image as a real-time ultrasound tomographic image representing the second observation cross section.

Another aspect of the present disclosure relates to a data processing method including: a step, which is executed by an ultrasound diagnostic apparatus in a first ultrasound examination on a subject, of extracting first cross section data corresponding to a first observation cross section from volume data acquired from an inside of the subject, generating and displaying a first past image as a tomographic image based on the first cross section data, and displaying a first current image as a real-time ultrasound tomographic image representing the first observation cross section; a step, which is executed by the ultrasound diagnostic apparatus in or after the first ultrasound examination, of generating an information file including positional information of the first observation cross section; and a step, which is executed by an ultrasound diagnostic apparatus that is the same as or different from the ultrasound diagnostic apparatus in a second ultrasound examination on the subject, of setting a second observation cross section for the volume data based on the positional information in the information file, extracting second cross section data corresponding to the second observation cross section from the volume data, generating and displaying a second past image based on the second cross section data, and displaying a second current image as a real-time ultrasound tomographic image representing the second observation cross section.

According to the present disclosure, it is possible to reduce the burden on the examiner who is the user in a case in which the past image and the current image are displayed in the ultrasound examination. Alternatively, according to the present disclosure, the correct past image is displayed in a case in which the past image and the current image are displayed in the ultrasound examination. Alternatively, according to the present disclosure, the information sharing is achieved among the plurality of ultrasound diagnostic apparatuses.

### BRIEF DESCRIPTION OF THE DRAWINGS

FIG. 1 is a block diagram showing a medical system according to an embodiment.

FIG. 2 is a diagram showing display of a past image and a current image.

FIG. 3 is a diagram showing an example of an information file.

FIG. 4 is a diagram showing a first example in which positional information is used.

FIG. 5 is a flowchart showing an operation example according to the embodiment.

FIG. 6 is a diagram showing a second example in which the positional information is used.

FIG. 7 is a diagram showing a third example in which the positional information is used.

### DESCRIPTION OF THE PREFERRED EMBODIMENTS

Hereinafter, an embodiment will be described with reference to the accompanying drawings.

### (1) Outline of Embodiment

A medical system according to the embodiment includes a first processing unit, a transmission unit, an acquisition unit, and a second processing unit. In the first ultrasound examination on a subject, the first processing unit extracts first cross section data corresponding to a first observation cross section from volume data acquired from an inside of the subject, and generates and displays a first past image as a tomographic image based on the first cross section data. In addition, the first processing unit displays a first current image as a real-time ultrasound tomographic image representing the first observation cross section. In or after the first ultrasound examination, the transmission unit transmits an information file including positional information of the first observation cross section to a server. In a second ultrasound examination after the first ultrasound examination, the acquisition unit acquires the information file from the server. In the second ultrasound examination, the second processing unit sets a second observation cross section for the volume data based on the positional information in the information file, extracts second cross section data corresponding to the second observation cross section from the volume data, and generates and displays a second past image based on the second cross section data. In addition, the second processing unit displays a second current image as a real-time ultrasound tomographic image representing the second observation cross section.

With the above-described configuration, in the second ultrasound examination after the first ultrasound examination, it is not necessary for a user to designate the second observation cross section, so that a burden on the user can be reduced. In addition, it is possible to correctly set the second observation cross section based on the positional information.

The first observation cross section and the second observation cross section are basically the same cross section. Similarly, the first cross section data and the second cross section data are basically the same cross section data. The first past image and the second past image are basically the same image. On the other hand, the first current image and the second current image are different images representing the same observation cross section (first observation cross section and second observation cross section). An ultrasound image formation unit, an image processing unit, and a display processing unit, which are described below, correspond to the first processing unit and the second processing unit. A communication unit described below corresponds to the transmission unit and the acquisition unit.

The volume data is generally acquired from the server. The volume data may be acquired from a storage unit in the ultrasound diagnostic apparatus. In the second ultrasound examination, the volume data may be acquired from the server together with the information file, the volume data may be acquired after the acquisition of the information file, or the volume data may be acquired before the acquisition of the information file. The medical system is generally a system including the ultrasound diagnostic apparatus and the server. The medical system may include a plurality of ultrasound diagnostic apparatuses. The medical system may be configured with a single ultrasound diagnostic apparatus.

In the embodiment, the information file includes volume data specification information for specifying the volume data. The acquisition unit acquires the volume data from the server based on the volume data specification information in the information file. With this configuration, it is easy to specify and acquire the volume data necessary for executing an image comparison function.

In the embodiment, the information file includes a thumbnail image pair generated based on the first past image and the first current image. The second processing unit displays the thumbnail image pair. The acquisition unit acquires the volume data in response to an operation on the thumbnail image pair. With this configuration, the volume data can be acquired after checking the past image to be displayed with reference to the thumbnail image pair. Only the thumbnail image representing an MR image may be displayed, and the thumbnail image may be selected by the user. In this case, MRI volume data corresponding to the thumbnail image is acquired.

The medical system according to the embodiment includes a first ultrasound diagnostic apparatus and a second ultrasound diagnostic apparatus. The first ultrasound diagnostic apparatus is connected to the server via a network, and comprises the first processing unit and the transmission unit. The second ultrasound diagnostic apparatus is connected to the server via the network, and comprises the acquisition unit and the second processing unit. The first ultrasound examination is performed by using the first ultrasound diagnostic apparatus, and the second ultrasound examination is performed by using the second ultrasound diagnostic apparatus.

With the above-described configuration, the information file can be shared among a plurality of ultrasound diagnostic apparatuses. Even in a case in which the ultrasound diagnostic apparatus used in the first ultrasound examination and the ultrasound diagnostic apparatus used in the second ultrasound examination are different from each other, a correct past image is displayed.

In the embodiment, the volume data is data acquired in a medical apparatus other than the ultrasound diagnostic apparatus. Each of the first past image and the second past image is a non-ultrasound image. The volume data is, for example, the volume data acquired by an MRI apparatus. The volume data is single 3D data or a collection of a plurality of 2D slice data. In the ultrasound diagnostic apparatus, the volume data for image processing may be reconstructed from original volume data.

In the embodiment, in the second ultrasound examination, the second processing unit generates and displays a reference image including a cross section model based on the positional information in the information file. In addition to the cross section model, the reference image may include a three-dimensional tissue image generated based on the volume data or a three-dimensional tissue image as a CG image. Displaying the reference image facilitates recognition of a position and an inclination of the observation cross section.

In the embodiment, the acquisition unit acquires a plurality of images, which are acquired in a plurality of ultrasound examinations and associated with the same positional information. The second processing unit displays a list of the plurality of images. With this configuration, for example, it is possible to display an image sequence representing a temporal change of an affected part or an effect of treatment on the affected part. The plurality of images are acquired from the server, acquired from a local memory in the ultrasound diagnostic apparatus, or acquired from the server and the local memory.

A data processing method according to the embodiment includes a first step, a second step, and a third step. The first step is a step executed by the ultrasound diagnostic apparatus in the first ultrasound examination of the subject. In the first step, the first cross section data corresponding to the first observation cross section is extracted from the volume data acquired from the inside of the subject, and the first past image as the tomographic image is generated and displayed based on the first cross section data. In addition, in the first step, the first current image as the real-time ultrasound tomographic image representing the first observation cross section is displayed. The second step is a step executed by the ultrasound diagnostic apparatus in or after the first ultrasound examination. In the second step, the information file including the positional information of the first observation cross section is generated. The third step is a step executed by an ultrasound diagnostic apparatus that is the same as or different from the above-described ultrasound diagnostic apparatus in the second ultrasound examination for the subject. In the third step, the second observation cross section is set for the volume data based on the positional information in the information file, the second cross section data corresponding to the second observation cross section is extracted from the volume data, and the second past image is generated and displayed based on the second cross section data. In addition, in the third step, the second current image as the real-time ultrasound tomographic image representing the second observation cross section is displayed.

The data processing method is a method performed on a single ultrasound diagnostic apparatus or a method performed over the plurality of ultrasound diagnostic apparatuses. A program for executing the data processing method is installed in an information processing apparatus via a portable storage medium or the network. The information processing apparatus includes a storage medium that non-temporarily stores the program. The information processing apparatus may be configured with one or a plurality of medical apparatuses.

### (2) Details of Embodiment

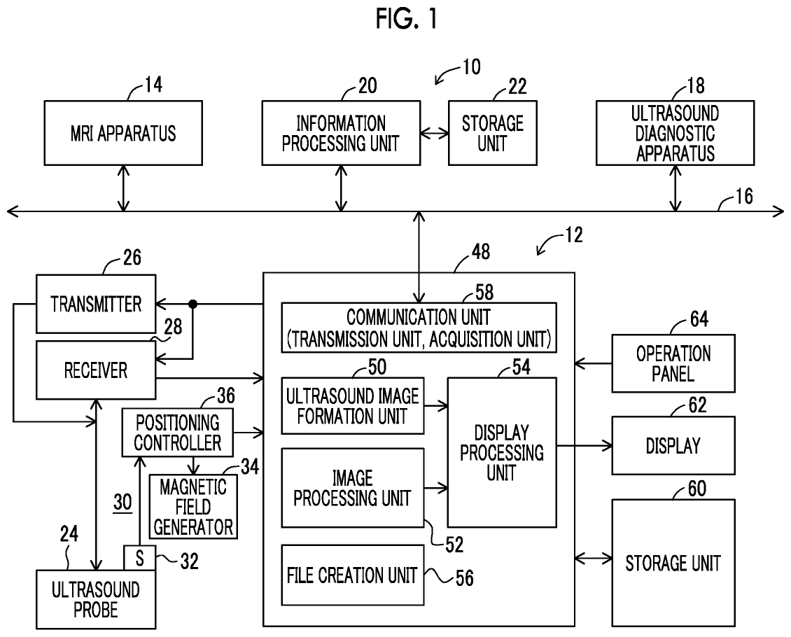

FIG. 1 shows the medical system according to the embodiment. The medical system is provided, for example, in a hospital. The shown medical system includes a server 10, an ultrasound diagnostic apparatus 12, and an MRI apparatus 14. The apparatuses 10, 12, and 14 are connected to a network 16. The medical system may include another ultrasound diagnostic apparatus 18.

The following description is made on the premise that an MRI examination on a mammary gland of a specific subject and a plurality of ultrasound examinations after the MRI examination. CT volume data may be acquired by performing a CT examination instead of the MRI examination, or ultrasound volume data may be acquired by performing an ultrasound examination instead of the MRI examination. The medical system shown in FIG. 1 can also be used in a case in which a site other than the mammary gland is an examination target.

The server 10 includes an information processing unit 20 and a storage unit 22. The information processing unit 20 includes a CPU executing a program. The storage unit 22 is configured with a semiconductor memory, a hard disk, or the like. The storage unit 22 stores the data acquired or generated by each apparatus. The server 10 is a server compliant with so-called digital imaging and communications in medicine (DICOM). Each data flowing on the network 16 is also data compliant with the DICOM. The server 10 and each data may be configured according to other standards.

The MRI apparatus 14 is used in the MRI examination. The MRI apparatus 14 acquires the volume data representing the inside of the subject (in the embodiment, an inside of a specific breast). The volume data is transmitted from the MM apparatus 14 to the server 10 via the network 16. The transmitted volume data is stored in the storage unit 22.

The ultrasound diagnostic apparatus 12 is used in the ultrasound examination. An ultrasound probe 24 is a device that transmits ultrasound and receives reflected waves. The ultrasound probe 24 (more precisely, a probe head in the ultrasound probe 24) is held by the user who is an examiner. A wave transmission/reception surface of the ultrasound probe 24 comes into contact with a surface of the subject (in the embodiment, a surface of the specific breast).

The ultrasound probe 24 includes a transducer array configured with a plurality of transducers. An ultrasound beam is formed by the transducer array, and electronic scanning with the ultrasound beam is performed. As the electronic scanning method, an electronic linear scanning method, an electronic sector scanning method, or the like is known. A beam scanning surface is formed in a body of the subject by the electronic scanning with the ultrasound beam. The ultrasound probe 24 may be provided with a two-dimensional transducer array consisting of a plurality of transducers arranged two-dimensionally. The ultrasound volume data may be acquired from the inside of the subject by using the configuration.

A transmitter 26 is an electronic circuit that functions as a transmission beam former. In the transmission, a plurality of transmission signals are output in parallel to the transducer array. As a result, a transmission beam is formed.

In the reception, a plurality of reception signals output in parallel from the transducer array are transmitted to a receiver 28. The receiver 28 is an electronic circuit that functions as a reception beam former. Specifically, the receiver 28 applies phasing addition to the plurality of reception signals, thereby generating reception beam data. A plurality of reception beam data arranged in an electronic scanning direction are generated for each electronic scanning with the ultrasound beam, thereby forming reception frame data. By repeating the electronic scanning with the ultrasound beam, a reception frame data sequence consisting of a plurality of reception frame data is generated. The reception frame data sequence is transmitted to an information processing unit 48.

The ultrasound diagnostic apparatus 12 according to the embodiment comprises a positioning system 30. The positioning system 30 includes a magnetic sensor 32, a magnetic field generator 34, and a positioning controller 36. The magnetic sensor 32 is fixed to the ultrasound probe 24. A special magnetic field for positioning is generated in the magnetic field generator 34 fixedly installed in an examination room. The magnetic field is detected by the magnetic sensor 32. The positioning controller 36 outputs a signal for generating the magnetic field to the magnetic field generator 34, and processes a detection signal from the magnetic sensor 32.

In the positioning controller 36 or the information processing unit 46, a position and a posture of the ultrasound probe 24 are specified based on the detection signal. That is, positional information for the ultrasound probe 24 is generated. The positional information includes information representing a position in an X-axis direction, information representing a position in a Y-axis direction, information representing a position in a Z-axis direction, information representing a rotation angle around an X-axis, information representing a rotation angle around a Y-axis, and information representing a rotation angle around a Z-axis. The substance of the positional information is a matrix.

In the embodiment, the positioning system 30 functions in at least the ultrasound examination of a first time (first ultrasound examination) on the subject. The positioning system 30 is not necessary for each of the ultrasound examinations of second and subsequent times. The processing of the positional information will be described in detail below.

A positioning system instead of the shown positioning system 30 may be used. For example, a positioning system using light or a positioning system having an acceleration sensor may be used. The positioning system may be excluded as long as the positional information can be specified by another method.

The information processing unit 48 functions as a controller and an operation unit. The information processing unit 48 is configured with a CPU executing a program. In FIG. 1, a part of the functions of the information processing unit 48 is represented by a plurality of blocks. Specifically, the information processing unit 48 functions as an ultrasound image formation unit 50, an image processing unit 52, a display processing unit 54, a file creation unit 56, and a communication unit 58.

The ultrasound image formation unit 50 generates the real-time ultrasound tomographic image (B-mode tomographic image) as a moving image based on the reception frame data sequence. The real-time ultrasound tomographic image is formed with a plurality of frames. Each frame corresponds to the tomographic image as a still image. The ultrasound image formation unit 50 includes a digital scan converter (DSC) having a coordinate transformation function and the like. The ultrasound image formation unit 50 may generate a blood flow image or the like as the real-time ultrasound image. Data representing the real-time ultrasound tomographic image is transmitted to the display processing unit 54. The real-time ultrasound tomographic image is displayed as the current image in execution of an image comparison mode.

The image processing unit 52 generates the tomographic image as the still image based on the cross section data cut out from the volume data in the execution of the image comparison mode. Specifically, the volume data is the MM volume data acquired from the server 10. In the first ultrasound examination, the observation cross section (first observation cross section) is designated for the MRI volume data by the user. The cross section data (first cross section data) corresponding to the observation cross section is cut out from the volume data, and the MR image as the tomographic image is generated based on the cross section data. The MR image is an image based on the volume data acquired in the past, and can be called a past image (first past image). Data representing the past image is output from the image processing unit 52 to the display processing unit 54.

In each of the ultrasound examinations of second and subsequent times (second ultrasound examination), in the execution of the image comparison mode, the image processing unit 52 automatically sets the observation cross section (second observation cross section) for the MRI volume data acquired from the server 10 based on the positional information acquired from the server 10, and cuts off the cross section data (second cross section data) corresponding to the observation cross section. Then, the image processing unit 52 generates the past image (second past image) which is the MR image based on the cross section data.

The image processing unit 52 according to the embodiment also has a function of generating the reference image including the cross section model and a function of generating the image sequence consisting of the plurality of images associated with the same positional information.

The display processing unit 54 generates an image to be displayed on a display 62. A signal representing the image is output to the display 62. The display processing unit 54 has a color operation function, an image composition function, and the like.

The file creation unit 56 generates the information file in the ultrasound examination of the first time (first ultrasound examination). The information file is transmitted to the server 10 by the communication unit 58 and saved in the server 10. In each of the ultrasound examinations of second and subsequent times (second ultrasound examination), the communication unit 58 acquires the information file from the server 10.

The information file has the volume data specification information and the positional information. The volume data specification information is information for specifying the volume data used in the ultrasound examination of the first time. The volume data is the MRI volume data in the embodiment, but may be another volume data. The positional information is information representing the position and the inclination of the observation cross section (first observation cross section) set for the volume data.

Specifically, in the execution of the image comparison mode, the position and the inclination of the observation cross section are adjusted with reference to the past image which is the displayed MR image such that the affected part is clearly imaged. As a result, the optimum observation cross section is set. Thereafter, the position and the posture of the ultrasound probe are adjusted by the user such that a content of the current image, which is the real-time ultrasound image, matches a content of the past image, which is the displayed MR image. In a state in which the contents of the two images match, for example, the user operates a specific button (freeze button, store button, or the like). The positional information at the time of the operation is registered in the information file.

It should be noted that calibration is performed as necessary before or after the start of the ultrasound examination. The calibration means the operation and the adjustment for matching a coordinate system included in the volume data with a coordinate system included in the positioning system.

The communication unit 58 exchanges data with and from other apparatuses. The communication unit 58 according to the embodiment functions as a transmission unit that transmits the information file to the server 10 in a case in which the information file is created in the ultrasound examination of the first time. The information file is saved in the storage unit 22 in the server 10. In addition, the communication unit 58 according to the embodiment functions as an acquisition unit that acquires the information file and the volume data from the server in each of the ultrasound examinations of second and subsequent times. The volume data to be acquired is specified based on the acquired information file, and the position and the inclination of the observation cross section to be set for the volume data are specified based on the acquired information file.

The ultrasound image formation unit 50, the image processing unit 52, and the display processing unit 54 function as the first processing unit and the second processing unit. The communication unit 58 functions as the transmission unit and the acquisition unit.

The real-time ultrasound tomographic image as the moving image is generally displayed on the display 62. In a case in which a freeze operation is performed, the still image is displayed as a freeze image. In the execution of the image comparison mode, the past image and the current image are simultaneously displayed in parallel. As described above, the past image is, for example, the MR image as the tomographic image, and the current image is the real-time ultrasound tomographic image, that is, the moving image. The display 62 is configured with, for example, an LCD or an organic EL display device.

An operation panel 64 is an input device, which includes a plurality of switches, a plurality of buttons, a track ball, a keyboard, and the like. In the first ultrasound examination, the position of the observation cross section is designated by the user using the operation panel 64. The storage unit 60 configures the local memory, which is configured with a semiconductor memory or a hard disk. The storage unit 60 stores a plurality of tomographic images generated by the transmission and the reception of the ultrasound, the information file downloaded from the server, the volume data downloaded from the server, and the like.

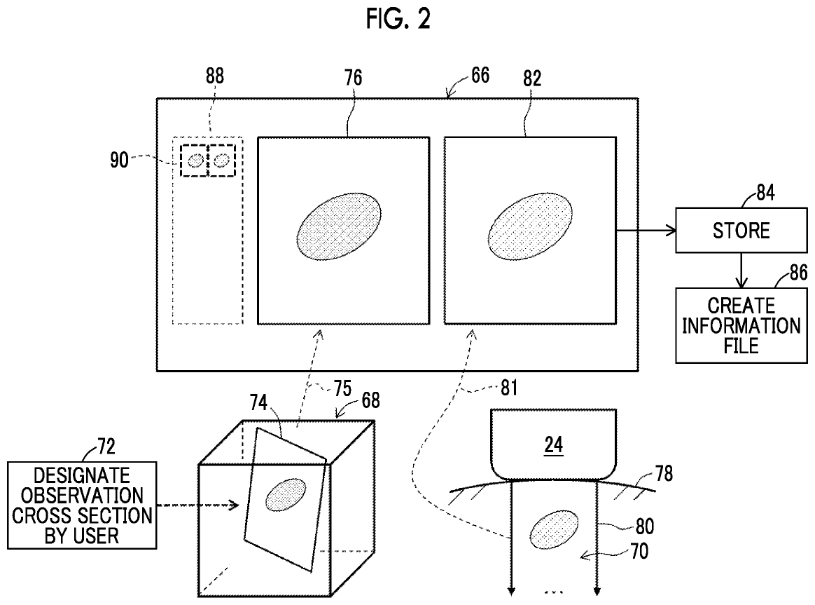

FIG. 2 shows an execution state of the image comparison mode in the ultrasound examination of the first time. An image 66 displayed on the display includes a past image 76 as the MR image and a current image 82 as the real-time ultrasound tomographic image. Volume data 68 is the MRI volume data acquired from the server. In a case in which the observation cross section is designated by the user (see reference numeral 72), the cross section data corresponding to an observation cross section 74 is cut out from the volume data 68. The past image 76 is generated and displayed based on the cross section data (see reference numeral 75).

In the ultrasound examination of the first time, the position and the inclination of the observation cross section 74 are adjusted by the user with reference to the past image 76 such that the affected part is clearly imaged in the past image 76. As a result, the optimum observation cross section is designated.

On the other hand, the ultrasound probe 24 comes into contact with a breast surface 78 of the subject, and electronic scanning with an ultrasound beam 80 is performed in that state. As a result, a beam scanning surface 70 is formed. The current image 82 is generated and displayed based on the frame data obtained from the beam scanning surface 70 (see reference numeral 81). In practice, the electronic scanning with the ultrasound beam 80 is repeated, and the current image 82 as a real-time moving image is displayed.

The position and the posture of the ultrasound probe 24 are adjusted by the user such that the content of the current image 82 matches the content of the past image 76 representing the optimum observation cross section. The store button is operated at a time when the two contents match (see reference numeral 84). As a result, the current image 82 (and the past image 76, as necessary) is saved in the local memory. Simultaneously, the information file is created (see reference numeral 86). The information file is transmitted to the server in or after the first ultrasound examination.

It should be noted that the freeze button may be operated before the operation of the store button. In this case, the positional information may be specified at the time of the operation of the freeze button. The thumbnail image pair is generated in a case in which the store button is operated. The thumbnail image pair consists of two low-resolution images (minified images) generated based on the past image and the current image. The thumbnail image pair is displayed in a thumbnail image display area 88 (see reference numeral 90). In addition, in the embodiment, the thumbnail image pair is inserted into the information file.

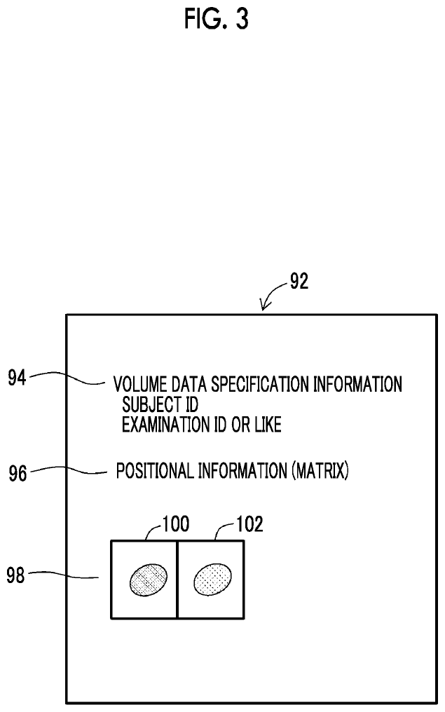

FIG. 3 shows an example of the information file. An information file 92 is created to be compliant with the DICOM. Of course, the information file 92 may be created in another format. The shown information file 92 includes volume data specification information 94 and positional information 96. In addition, the information file 92 includes a thumbnail image pair 98.

The volume data specification information 94 includes a subject ID (patient ID), an examination ID (study instance ID), and the like. The subject ID is used as a search key in searching for the information file or the volume data. The positional information 96 is information generated by the positioning system, and the substance thereof is a matrix consisting of a plurality of coordinate values. The thumbnail image pair 98 is formed with a thumbnail image 100 representing the past image (MR image) and a thumbnail image 102 representing the current image (stored ultrasound tomographic image).

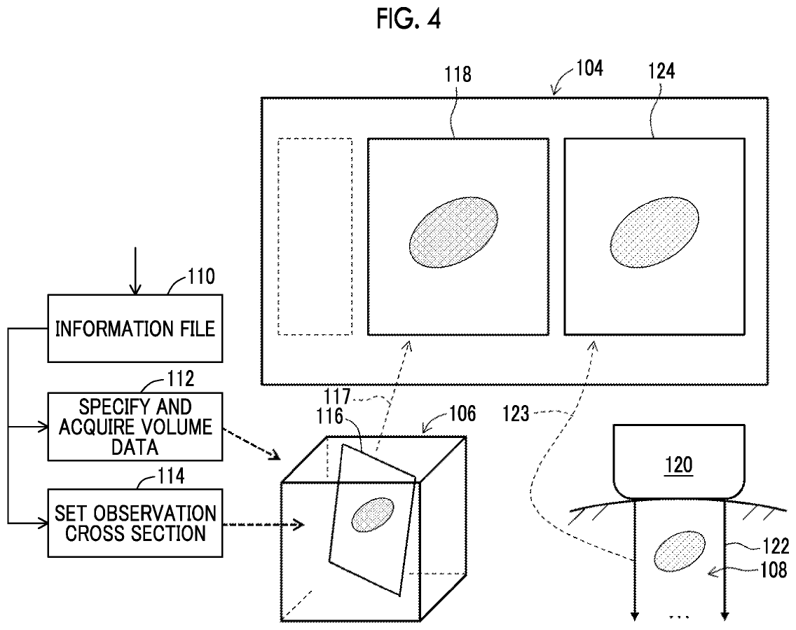

FIG. 4 shows an execution state of the image comparison mode in the ultrasound examination of the second time. FIG. 4 also shows a first example in which the positional information is used. In the shown example, for example, the information file is searched for and specified by using the subject ID, and the information file is acquired from the server (see reference numeral 110). The volume data is specified with reference to the information file, and then the volume data is acquired from the server. In FIG. 4, the acquired volume data is denoted by reference numeral 106. On the other hand, the positional information of the observation cross section is specified with reference to the acquired information file, and an observation cross section 116 is automatically set for the volume data 106 based on the positional information (see reference numeral 114).

An image 104 displayed on the display includes a past image 118 and a current image 124. The past image 118 is an image generated based on the cross section data corresponding to the observation cross section 116 (see reference numeral 116). The current image 124 is an image acquired by using the ultrasound probe 120, and more specifically, the real-time ultrasound tomographic image as the moving image based on the frame data sequence generated by repeating the electronic scanning with the ultrasound beam 122.

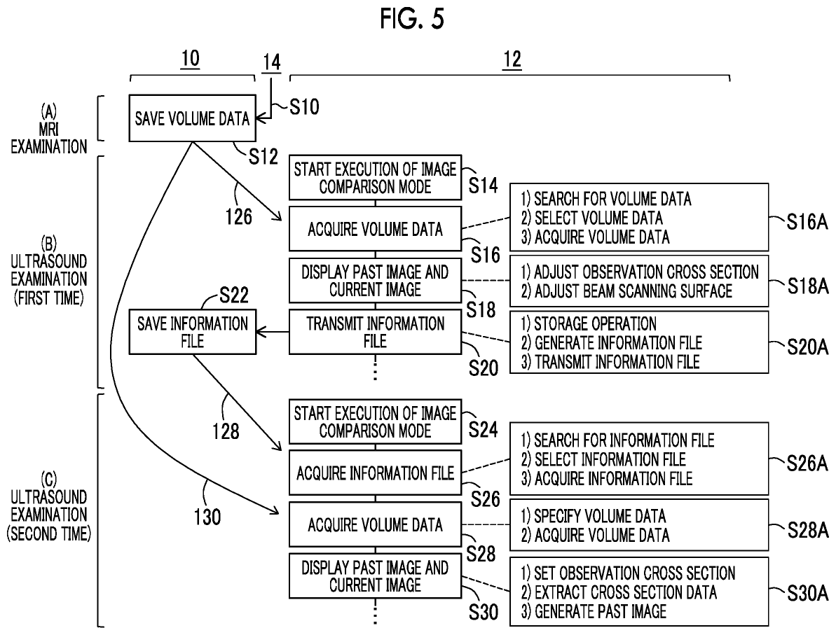

FIG. 5 shows an operation example of the medical system according to the embodiment. The medical system includes the server 10, the ultrasound diagnostic apparatus 12, and the MRI apparatus 14. (A) shows the MRI examination on the subject. (B) and (C) show the ultrasound examinations of the first time and the second time for the subject. The content of the performance in the ultrasound examination of the second time and the content of the performance in each ultrasound examination of the third and subsequent times are basically the same.

In S10, the MM examination is performed, and thus the volume data is acquired from the inside of the subject. In S12, the volume data is transmitted to the server 10, and the transmitted volume data is saved in the server 10.

In the ultrasound examination of the first time, the execution of the image comparison mode is started in S14, and the volume data is transmitted from the server 10 to the ultrasound diagnostic apparatus 12 in S16 (see reference numeral 126). Specifically, as shown in S 16A, first, a search condition is designated by the user, and the volume data saved in the server is searched for, in accordance with the search condition. A search result is displayed on the ultrasound diagnostic apparatus 12. The volume data is selected by the user from the search result. As a result, the selected volume data is transmitted from the server 10 to the ultrasound diagnostic apparatus 12, that is, the selected volume data is acquired in the ultrasound diagnostic apparatus 12.

Subsequently, in S18, the past image as the MR image and the current image as the real-time ultrasound tomographic image are simultaneously displayed in parallel. Specifically, as shown in S 18A, the position and the inclination of the observation cross section are adjusted with reference to the past image by the user such that the affected part is clearly imaged. Thereafter, the position and the posture of the ultrasound probe (that is, the beam scanning surface) are adjusted such that the content of the current image matches the content of the past image.

In S20, the information file is generated, and the generated information file is transmitted. Specifically, as shown in 520A, a storage operation is performed in a state in which the content of the past image and the content of the current image match, and thus the current image is saved. The information file is generated accordingly. The information file includes the volume data specification information, the positional information, the thumbnail image pair, and the like. The information file is transmitted from the ultrasound diagnostic apparatus 12 to the server 10. In S22, the information file is saved in the server 10.

In the ultrasound examination of the second time, in S24, the execution of the image comparison mode is started. In S26, the information file is acquired. Specifically, as shown in S26A, the information file is searched for on the server 10 based on, for example, the subject ID, and thus the search result is displayed on the ultrasound diagnostic apparatus 12. The user selects a specific information file from the search result. The selected information file is transmitted from the server 10 to the ultrasound diagnostic apparatus 12 (see reference numeral 128). As a result, the ultrasound diagnostic apparatus 12 acquires the information file.

In S28, the volume data is acquired based on the information file. Specifically, with reference to the volume data specification information in the information file, the volume data to be acquired is specified, and the volume data is transmitted from the server 10 to the ultrasound diagnostic apparatus 12 (see reference numeral 130).

In the embodiment, the thumbnail image pair included in the information file is displayed after the acquisition of the information file. The volume data associated with the thumbnail image pair is acquired by the user's selection of the displayed thumbnail image pair.

Subsequently, in S30, the past image and the current image are simultaneously displayed in parallel. Specifically, as shown in reference numeral 30A, the position and the inclination of the observation cross section in the volume data are specified based on the positional information in the information file, and the cross section data corresponding to the observation cross section is extracted from the volume data. The MR image as the past image is generated based on the cross section data, and the generated MR image is displayed.

As described above, according to the embodiment, the information file is created in the ultrasound examination of the first time. By acquiring and referring to the information file in the ultrasound examinations of the second and subsequent times, the volume data can be selected without an error, and the observation cross section can be appropriately set.

In the above description, the ultrasound diagnostic apparatus used in the first ultrasound examination and the ultrasound diagnostic apparatus used in the second ultrasound examination are the same, but the same advantage is obtained in a case in which the ultrasound diagnostic apparatus used in the first ultrasound examination and the ultrasound diagnostic apparatus used in the second ultrasound examination are different from each other. That is, according to the technique of the embodiment, it is possible to share the information file among the plurality of ultrasound diagnostic apparatuses.

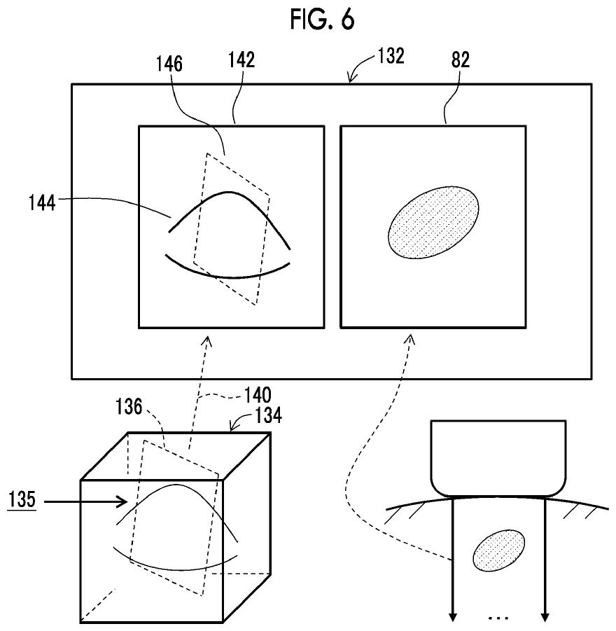

FIG. 6 shows a second example in which the positional information is used. An image 132 includes a reference image 142 and the current image 82. The current image is the real-time ultrasound tomographic image. The reference image 142 includes a three-dimensional tissue image (tissue model) 144 representing the tissue and a cross section model 146 representing the observation cross section.

The three-dimensional tissue image 144 is generated based on volume data 134. For example, the three-dimensional tissue image 144 is generated by volume rendering on the volume data 134. The three-dimensional tissue image 144 may be formed with a schematic three-dimensional image. The cross section model 146 is generated based on a position and an inclination of an observation cross section 136 set for the volume data 134 (see reference numeral 140). The cross section model 146 corresponds to a computer graphic, which is a figure. The position and the inclination of the observation cross section 136 are specified by the positional information in the information file (see reference numeral 135).

By observing the reference image 142, the position of the observation cross section can be easily specified in relation to the tissue. The reference image 142 may be displayed together with the past image and the current image 82.

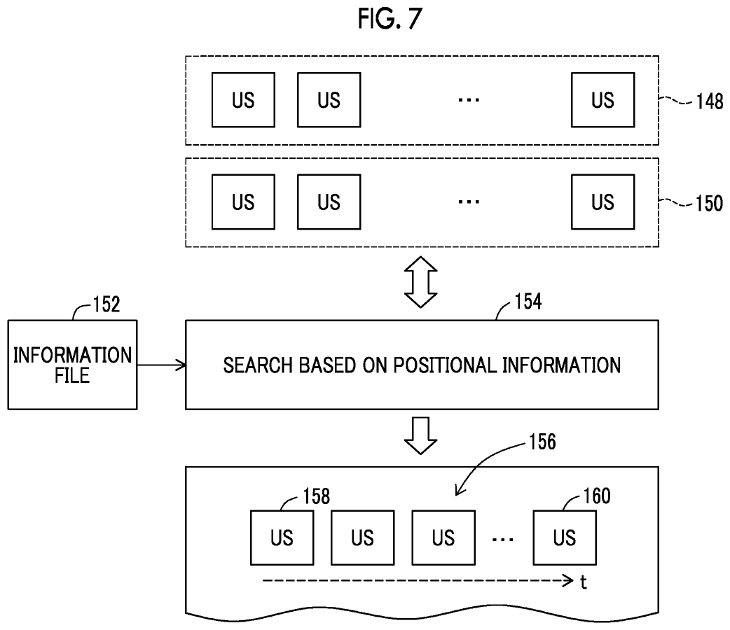

FIG. 7 shows a third example in which the positional information is used. A plurality of saved ultrasound images are stored in the storage unit 148 in the server, and the plurality of saved ultrasound images are also stored in the storage unit 150 in the ultrasound diagnostic apparatus. The positional information is associated with each ultrasound image.

As shown in reference numeral 154, the image is searched for based on the positional information in an information file 152. Specifically, a plurality of images associated with the same positional information are searched for. A shown image sequence 156 is formed with a plurality of images 158 and 160, and the plurality of images 158 and 160 are associated with the same positional information. The plurality of images 158 and 160 are arranged in time series, that is, in an order of examination date. With the display of the image sequence 156, the temporal change of the affected part can be easily recognized, or the effect of treatment can be easily recognized on a time axis.

What is claimed is:

1. A medical system comprising:
a first processing unit configured to, in a first ultrasound examination on a subject, extract first cross section data corresponding to a first observation cross section from volume data acquired from an inside of the subject, generate and display a first past image as a tomographic image based on the first cross section data, and display a first current image as a real-time ultrasound tomographic image representing the first observation cross section;
a transmission unit configured to, in or after the first ultrasound examination, transmit an information file including positional information of the first observation cross section to a server;
an acquisition unit configured to, in a second ultrasound examination after the first ultrasound examination, acquire the information file from the server; and
a second processing unit configured to, in the second ultrasound examination, set a second observation cross section for the volume data based on the positional information in the information file, extract second cross section data corresponding to the second observation cross section from the volume data, generate and display a second past image based on the second cross section data, and display a second current image as a real-time ultrasound tomographic image representing the second observation cross section.

2. The medical system according to claim 1,
wherein volume data specification information for specifying the volume data is included in the information file, and
the acquisition unit is configured to acquire the volume data from the server based on the volume data specification information in the information file.

3. The medical system according to claim 1,
wherein a thumbnail image pair generated based on the first past image and the first current image is included in the information file,
the second processing unit is configured to display the thumbnail image pair, and
the acquisition unit is configured to acquire the volume data in response to an operation on the thumbnail image pair.

4. The medical system according to claim 1, further comprising:
a first ultrasound diagnostic apparatus connected to the server via a network and including the first processing unit and the transmission unit; and
a second ultrasound diagnostic apparatus connected to the server via the network and including the acquisition unit and the second processing unit,
wherein the first ultrasound examination is performed by using the first ultrasound diagnostic apparatus, and
the second ultrasound examination is performed by using the second ultrasound diagnostic apparatus.

5. The medical system according to claim 1,
wherein the volume data is acquired in a medical apparatus other than an ultrasound diagnostic apparatus, and
each of the first past image and the second past image is a non-ultrasound image.

6. The medical system according to claim 1,
wherein the second processing unit is configured to, in the second ultrasound examination, generate and display a reference image including a cross section model based on the positional information in the information file.

7. The medical system according to claim 1,
wherein the acquisition unit is configured to acquire a plurality of images, which are acquired in a plurality of ultrasound examinations and associated with the same positional information, and
the second processing unit is configured to display a list of the plurality of images.

8. A data processing method comprising:
a step, which is executed by an ultrasound diagnostic apparatus in a first ultrasound examination on a subject, of extracting first cross section data corresponding to a first observation cross section from volume data acquired from an inside of the subject, generating and displaying a first past image as a tomographic image based on the first cross section data, and displaying a first current image as a real-time ultrasound tomographic image representing the first observation cross section;
a step, which is executed by the ultrasound diagnostic apparatus in or after the first ultrasound examination, of generating an information file including positional information of the first observation cross section; and
a step, which is executed by an ultrasound diagnostic apparatus that is the same as or different from the ultrasound diagnostic apparatus in a second ultrasound examination on the subject, of setting a second observation cross section for the volume data based on the positional information in the information file, extracting second cross section data corresponding to the second observation cross section from the volume data, generating and displaying a second past image based on the second cross section data, and displaying a second current image as a real-time ultrasound tomographic image representing the second observation cross section.

9. A non-transitory storage medium storing a program executed in an information processing apparatus, the program comprising:
a function of, in a first ultrasound examination on a subject, extracting first cross section data corresponding to a first observation cross section from volume data acquired from an inside of the subject, generating and displaying a first past image as a tomographic image based on the first cross section data, and displaying a first current image as a real-time ultrasound tomographic image representing the first observation cross section;
a function of, in or after the first ultrasound examination, generating an information file including positional information of the first observation cross section; and
a function of, in a second ultrasound examination on the subject, setting a second observation cross section for the volume data based on the positional information in the information file, extracting second cross section data corresponding to the second observation cross section from the volume data, generating and displaying a second past image based on the second cross section data, and displaying a second current image as a real-time ultrasound tomographic image representing the second observation cross section.
